(19) United States
(12) Patent Application Publication
(10) Pub. No.: US 2008/0119819 A1
(43) Pub. Date: May 22, 2008

(54) Use of Growth Hormone in Treatment of Long-Bone Fractures

(76) Inventors: Michael Hojby, Kongens Lyngby (DK); Mapoko Mbelenge IIondo, Vallensbaek (DK); Kirsten Raun, Lyngby (DK)

Correspondence Address: NOVO NORDISK, INC.;INTELLECTUAL PROPERTY DEPARTMENT, 100 COLLEGE ROAD WEST, PRINCETON, NJ, 08540, US

(21) Appl. No.: 11/579,219

(22) PCT Filed: Apr. 29, 2005
(86) PCT No.: PCT/EP05/51966

§ 371 Date: Sep. 19, 2007

(30) Foreign Application Priority Data
Apr. 30, 2004 (PA) 2004 00691

(51) Int. Cl. A61K 38/27; A61M 31/00

(52) U.S. Cl. 604/500; 530/399

(57) ABSTRACT

A method for promoting healing of a long-bone fracture in a human subject in need thereof comprises administration, e.g. by subcutaneous injection, of human growth hormone (hGH), or an analogue or derivative thereof, to the subject in a dose equivalent to 10 45-100 μg hGH/kg body weight/day.

### FIELD OF THE INVENTION

The present invention relates to the use of human growth hormone, or analogues or derivatives thereof, for promoting healing of long-bone fractures in human subjects.

### BACKGROUND OF THE INVENTION

There are numerous publications relating to the effect of growth hormone on fracture healing in non-human mammals. Reports of studies relating to rats include the following:

Ashton et al. [Br. J. Exp. Pathol. 64, 479 (1983)] and Tylkowski et al. [Clin. Orthop. 115, 274 (1976)] disclose the healing of rat tibiae by administering growth hormone, the tibiae healing with increased strength.

Jørgensen et al. [Calcified Tissue 44 (1989)], abstract D20] describe the injection of rats with human growth hormone (hGH). The mechanical strength of femur and tibia was measured. The maximum stiffness of the fracture was increased.

Bak et al. [Bone 11, 233 (1990)] describe the effect of hGH on the biomechanical properties of healing rat-tibial fractures. After 40 days of healing, the maximum stiffness of the fractures had increased.

Nielsen et al. [Acta Orthop. Scand. 62(3), 244-247 (1991)] describe the promoting effect of growth hormone on tibial fractures in rats. It is concluded that GH stimulates longitudinal bone growth by stimulating cell differentiation in the geminal zone of the growth plate. The maximum stiffness of the fractures increased in rats injected with growth hormone for two or three weeks.

Mosekilde et al. (Bone and Mineral, The XIth International Conference on Calcium Regulating Hormones, Florence, Italy, Apr. 24-29, 1992, abstract No. 504) describe the long-term effect of growth hormone on healing of rat tibial fractures. The results revealed an initial stimulatory effect of GH on callus formation, but the callus was loosely structured with significantly lower trabecular bone volume compared to untreated rats.

Mosekilde et al. [Bone 14, 19-27 (1993)] describe the effects of growth hormone on fracture healing in rats. On the basis of this study it was concluded that although there was an initially stimulatory effect of growth hormone on callus formation, the callus formed during growth hormone treatment was abnormal with an extremely loose structure, and modelling and remodelling of this callus were delayed. It appeared that the bone marrow cells grew at the expense of the mineralised callus tissue, or that the normal architecture of the callus tissue was disrupted.

Castillo et al. [Hormone Research, 33^{rd} Annual Meeting of the European Society for Paediatric Endocrinology (ESPE), Maastricht, Jun. 22-25, 1994, abstract No. 360] describe the stimulating effect of human growth hormone on fracture healing in rats. Recombinant hGH therapy accelerated healing of femur fractures in rats at 12 and 18 days post-fracture. It appeared that hGH acted on skeletal tissues directly by stimulating stem cells and indirectly by stimulating the production of local IGF-1.

In the case of rabbits, Herold et al. [Acta Orthop. Scand. 42, 377-384 (1971)] concluded that neither bovine nor porcine growth hormone administered intramuscularly produced any significant acceleration of healing of long-bone fractures.

Carpenter et al. [Journal of Bone and Joint Surgery, 74-A(3), 359 (1992)] describe the failure of growth hormone to alter the biomechanics of fracture-healing in rabbits, and according to this study, administration of growth hormone had no effect on fracture healing.

A report on a study with dogs by Buonomo et al. [10^{th} International Congress of Endocrinology, Jun. 12-13, 1996, San Francisco, USA, abstract No. P1-576 from Program & Abstracts (Vol. I: June 12-13)] describes the metabolic effect of canine somatotropin (cST), i.e. canine growth hormone, on bone growth factors and fracture healing in dogs. Treated dogs showed a 3- and 5-fold increase in strength and stiffness, respectively, of the healing fracture compared to untreated dogs.

There appear to have been relatively few systematic studies of the influence of growth hormone treatment on fracture healing in humans. In an early study reported by Koskinen [Acta Orthop. Scand., Suppl. 62, Munksgaard, Copenhagen (1963)], 60 adult patients aged 18-78 years having femoral, tibial or humeral fractures were treated with chromatographically purified porcine growth hormone (10 U.S.P. units), isolated from pig hypophysis, in combination with thyrotropin (thyroid stimulating hormone, TSH) (2 U.S.P units) administered intramuscularly every second day. The normal period of hormone administration was 3-10 weeks, although longer treatment times were employed in cases with non-union or delayed union, and with compound fractures or osseous defect. The overall conclusion from this study was that the pig growth hormone/TSH combination treatment had an osseous anabolic effect, and that administration of the two hormones was beneficial as an aid to appropriate orthopaedic treatment, promoting the healing of fractures and shortening the time required to achieve osseous consolidation, especially in cases in which delayed union was anticipated or non-union was an established fact.

Koskinen et al. [Med. Welt 26, 1905-1910 (1975)] have reported a study of the effects of human growth hormone (hGH) treatment in 20 patients aged 16-58 years admitted to hospital with a poor healing prognosis because of delayed union or non-union of a fracture of a long bone (tibia, femur or humerus). The hGH was administered intramuscularly in a dose of 16 I.U. every second day. Bone union was reported to occur in all 20 hGH-treated patients.

Lindholm et al. [Hormon. Metab. Res. 9, 245-246 (1977)] have reported a study of the effects of hGH treatment in 12 patients with “fresh fractures of the lower leg”. The hGH was administered intramuscularly in a dose of 16 I.U. every second day during 5 weeks, in addition to orthopaedic treatment. It appears to have been concluded that the results added nothing new in relation to the results published previously by Koskinen et al. in Med. Welt (vide supra). No harmful side effects of the administration of hGH were encountered.

WO 91/11148 discloses, inter alia, a method for local administration of a biologically active substance enhancing the healing of bone fractures or of a bone and a prosthesis to be united wherein the biologically active substance (e.g. hGH) is administered directly to the bone surfaces to be healed or to the interface between a bone and a prosthesis which are to be united.

There appears to be a general paucity of data on which to base any reliable conclusions concerning the effect of administration of human growth hormone to human subjects with long-bone fractures, particularly acute and sub-acute long-bone fractures (vide infra), and the study underlying the present invention was initiated in order to remedy this situation.

### SUMMARY OF THE INVENTION

The present invention thus relates, inter alia, to a method of promoting healing of a long-bone fracture in a human subject, the method comprising administering human growth hormone (hGH), or an analogue (variant) or derivative thereof, to the subject in a dose equivalent to 45-100 μg hGH/kg body weight/day.

### DETAILED DESCRIPTION OF THE INVENTION

As outlined above, one aspect of the present invention relates to a method of promoting healing of a long-bone fracture in a human subject (i.e. a human subject in need thereof), the method comprising administering human growth hormone (hGH), or an analogue or derivative thereof (vide infra), to the subject in a dose equivalent to 45-100 μg hGH/kg body weight/day. In particular embodiments of the method, parenteral administration in the form of subcutaneous administration (e.g. by injection) of hGH, or of an hGH analogue or hGH derivative, is employed, but other relevant routes of administration include alternative parenteral routes, such as via intramuscular, intraperitoneal or intravenous injection, or by implant.

A further aspect of the invention relates to the use of human growth hormone (hGH), or an analogue or derivative thereof, for the manufacture of a medicament which is to be administered (suitably subcutaneously) to a human subject in a dose equivalent to 45-100 μg hGH/kg body weight/day for promoting healing of a long-bone fracture in the subject.

A still further aspect of the invention relates to the use of human growth hormone (hGH), or an analogue or derivative thereof, for the manufacture of a medicament for treating a human subject so as to promote healing of a long-bone fracture in the subject, the treatment comprising administering the medicament (suitably subcutaneously) to the subject in a dose equivalent to 45-100 μg hGH/kg body weight/day.

The term “promoting” in relation to fracture healing in the context of the invention refers to increasing the likelihood of achieving union (successful healing) of the fracture and/or to accelerating the rate of healing of the fracture.

In another aspect of the invention, the human subject in question is an an adult. In the context of the present invention the term “adult” indicates a male or female person aged 18 years or more, in that human skeletal growth and development is normally complete, or substantially complete, by the age of 18 years.

Long bones are hard, dense bones that provide strength, structure and mobility [e.g. the femur (thigh bone)]. A long bone has a shaft and two ends. There are also bones in the fingers that are regarded as “long bones” owing to their shape, even though they are relatively short in length. Long bones contain yellow bone marrow and red bone marrow. The term “long-bone fracture” as employed in the present context thus includes, in particular, but is not limited to, a fracture of one or more of the bones of the upper or lower leg or arm of the subject (i.e. the femur, the tibia and the fibula of a leg, and the humerus, the ulna or the radius of an arm).

The methodology of the invention is well suited for the treatment of acute or sub-acute long-bone fractures. “Acute” fractures are fractures of recent occurrence, and in the context of the present invention are fractures that have occurred at most 6 days prior to commencement of treatment in accordance with the invention, and for which at most 3 days have elapsed since surgical or other primary fixation of the fracture (e.g. by intramedullary nailing and/or by plaster cast fixation). Sub-acute fractures in the context of the invention are fractures that are of less recent occurrence (i.e. that have occurred more than 6 days days prior to commencement of treatment in the manner of the invention) and have been subjected to primary fixation of the fracture, but for which invasive secondary procedures (such as replacement of an intramedullary nail, change of fixation method, or bone grafting) have not yet been found necessary.

Both closed and open long-bone fractures may be treated in the manner of the invention. “Closed” fractures in the context of the invention are fractures that have not been attended by soft-tissue damage to the extent that there has been exposure of the bone to the external environment. “Open” fractures in the context of the invention are fractures in which the bone has been exposed to the outside environment (e.g. as a result of abrasion or penetration of the skin and underlying tissues).

Closed tibia fractures are frequently classified in accordance with the Tscherne classification system (Table 1, below), whilst open tibia fractures are often classified in accordance with the Gustilo classification system (Table 2, below).

TABLE 1
Tscherne classification of closed fractures

| Type | Description |
|---|---|
| Type C0 | Simple fracture with little or no soft |
| | tissue injury |
| Type C1 | Superficial abrasion, mild to moderately |
| | severe fracture configuration |
| Type C2 | Deep contamination with local skin or |
| | muscle contusion. Moderately severe |
| | fracture configuration |
| Type C3 | Extensive contusion or crushing of skin |
| | or destruction of muscle. Severe fracture |

TABLE 2
Gustilo classification of open fractures

| Type | Description |
|---|---|
| Type I | Clean wound of <1 cm in length |
| Type II | Wound >1 cm in length without extensive |
| | soft tissue damage |
| Type III | Wound usually >5 cm associated with |
| | extensive soft tissue damage |
| Type IIIA | Adequate periosteal cover |
| Type IIIB | Significant periosteal stripping |
| Type IIIC | Vascular repair required to re-vascularise |
| | leg |

The methodology of the present invention is believed to be of value in the treatment of closed and open long-bone fractures within all of the classification categories in question.

Relatively frequently occurring long-bone fractures that are well suited for treatment in the manner according to the invention include both closed and open fractures of the femur, tibia or fibula of the leg, and of the humerus, ulna or radius of the arm.

As already indicated to some extent, not only human growth hormone (hGH) per se, but also analogues, variants or derivatives of hGH (including any combination of hGH and analogues, variants or derivatives of hGH), may be employed in the context of the invention. The term “human growth hormone” (hGH; sometimes also referred to, inter alia, as “somatropin” or “somatotropin”) is generally understood to refer to the protein (polypeptide) hormone consisting of a single chain of 191 amino acid residues cross-linked by two disulfide bridges, the monomeric form thereof having a molecular weight of approx. 22000 (22 kDa).

Growth hormone preparations isolated from human pituitary are not homogeneous. For example, a smaller (20 kDa) variant produced from the same gene is also known. The “basic hGH” variant (hGH-V) expressed by the placenta during pregnancy is another analogue/variant which is a product of a separate gene; like the 22 kDa hGH polypeptide it consists of 191 amino acid residues, but 13 amino acid residues at various positions in the sequence differ from those in 22 kDa hGH [see, e.g, Bewley et al., Adv. Enzymol. 42, 73-166 (1975), and Frankenne et al., J. Clin. Endocrin. and Metabol. 66, 1171-1180 (1988)].

Apart from the above-mentioned particular analogues or variants of hGH, analogues or variants of hGH, in general, in the context of the present invention encompass: truncated forms of hGH, i.e. truncated forms of hGH wherein one or more amino acid residues has/have been deleted; hGH analogues wherein one or more amino acid residues in hGH has/have been substituted with another amino acid residue, preferably a residue of a naturally occurring amino acid, as long as the substitution does not lead to any adverse effect such as antigenicity or significantly reduced activity; and N- and/or C-terminally extended forms of hGH (such as Met-hGH, Met-Glu-Ala-Glu-hGH or Ala-Glu-hGH).

Other examples of hGH analogues of relevance in relation to the present invention include hGH analogues wherein all four of the amino acid residues at positions 172, 174, 176 and 178, respectively, are replaced by a set of four amino acid residues (in the given respective order) chosen among the following:
(R, S, F, R);
(R, A, Y, R);
(K, T, Y, K);
(R, S, Y, R);
(K, A, Y, R);
(R, F, F, R);
(K, Q, Y, R);
(R, T, Y, H);
(Q, R, Y, R);
(K, K, Y, K);
(R, S, F, S) and
(K, S, N, R);
   as disclosed in WO 92/09690.

Additional examples of hGH analogues of relevance in relation to the present invention include hGH with a set of substitutions chosen among the following:
R167N, D171S, E174S, F176Y and I179T;
R176E, D171S, E174S and F176Y;
F10A, M14W, H18D and H21N;
F10A, M14W, H18D, H21N, R167N, D171S, E174S, F176Y and I179T;
F10A, M14W, H18D, H21N, R167N, D171A, E174S, F176Y and I179T;
F10H, M14G, H18N and H21N;
F10A, M14W, H18D, H21N, R167N, D171A, T175T and I179T; and
F10I, M14Q, H18E, R167N, D171S and I179T;
   as disclosed in U.S. Pat. No. 6,143,523.

Further examples of hGH analogues of relevance in relation to the present invention include hGH with the following set of substitutions:
H18A, Q22A, F25A, D26A, Q29A, E65A, K168A and E174A and wherein G120 is further substituted with another amino acid residue, e.g. R, K, W, Y, F or E; as disclosed in U.S. Pat. No. 6,004,931.

Still further examples of hGH analogues of relevance in relation to the present invention include hGH with the following sets of substitutions:
H18D, H21N, R167N, K168A, D1715, K172R, E174S and I179T, as disclosed in U.S. Pat. No. 5,849,535;
H18D, H21N, R167N, K168A, D171S, K172R, E1745 and 1179T, and wherein G120 is further substituted with another amino acid residue, e.g. R, K, W, Y, F or E, as disclosed in U.S. Pat. No. 6,057,292;
H18D, H21D, R167N, K168A, D171S, K172R, E174S and I179T; or
H18A, Q22A, F25A, D26A, Q29A, E65A, K168A and E174A, as disclosed in WO 97/11178; and
   hGH with a set of substitutions chosen among the following:
K168A and E174A;
R178N and I179M; and
K172A and F176A,
   as disclosed in WO 90/04788.

Derivatives of hGH in the context of the invention encompass, for example, deamidated or sulfoxidated forms of hGH, as well as dimers and higher oligomers of hGH. Other hGH derivatives of relevance include those in which hGH is conjugated to a molecule such as an albumin, e.g. human serum albumin (see, e.g., WO 97/24445), or a water-soluble polymer, such as a polyethyleneglycol (PEG) (see, e.g., WO 03/044056), in order to achieve, e.g., protracted duration of GH activity.

It is further contemplated that administration of insulin-like growth factor 1 (IGF-1) or an analogue or variant thereof (defined analogously to analogues and variants of hGH; vide supra) and/or a complex thereof with an IGF-binding protein (such as IGFBP-3) may be employed as an alternative to administration of hGH or an hGH analogue or hGH derivative (or a combination thereof) in the manner of the invention.

As already indicated, the administered hGH, or the administered analogue or derivative of hGH, is administered in a dose equivalent to 45-100 μg hGH/kg body weight/day (calculated as μg of pure 22 kDa hGH polypeptide). In the case of administration of hGH per se, it is clear that this may in principle be achieved, for example, by administering a dose in the range 45-100 μg hGH/kg body weight once daily, a dose in the range 90-200 μg hGH/kg body weight once every second day, a dose in the range 135-300 μg hGH/kg body weight once every third day . . . and so on. An alternative possibility will be administration of multiple doses each day, e.g. administration of a dose in the range 22.5-50 μg hGH/kg body weight twice daily, a dose in the range 15-33.3 μg hGH/kg body weight three times daily, . . . and so on. The choice of dosing regimen will be influenced, among other things, by safety considerations (safety associated with administration of a given single dose) and by considerations relating to acceptability on the part of the treated subject (e.g. level of inconvenience, and acceptable frequency of injections, e.g. subcutaneous injections), tempered by considerations relating to optimal maintenance of a therapeutically beneficial level of hGH in the organism during the period of treatment according to the invention.

In the case of administration of an hGH analogue or hGH derivative, important factors to be taken into account in determining the dose of the analogue or derivative which is substantially equivalent to a given dose of hGH per se will include (a) the relative therapeutic efficacy of the analogue or derivative in question (on a molar basis relative to that of hGH per se) as a substitute for hGH per se, and (b) the ratio between the molecular weight of the analogue or derivative in question and that of hGH per se.

In a particular aspect of the invention, the administration of hGH (or of an analogue or derivative thereof) to the subject to be treated in the manner of the invention takes place once daily.

In a further aspect of a method according to the invention, human growth hormone (hGH) per se, i.e. 22 kDa hGH, is administered to the subject in question.

Human growth hormone (hGH) compositions may be in a form suited for systemic injection or infusion, and may, as such, be formulated with a suitable liquid vehicle, such as sterile water or an isotonic saline or glucose solution. The compositions may be sterilized by conventional sterilization techniques which are well known in the art. The resulting aqueous solutions may be packaged for use as such, or they may be filtered under aseptic conditions and lyophilized, the lyophilized preparation being combined with the appropriate sterile aqueous vehicle prior to administration. The composition may contain pharmaceutically acceptable auxiliary substances as required to approximate physiological conditions, such as buffering agents, tonicity-adjusting agents and the like. Non-limiting examples of buffering agents include citrate salts, phosphate salts and histidine; non-limiting examples of tonicity adjusting agents include sugars, such as sucrose and mannitol, and salts, such as alkali metal and alkaline earth metal chlorides, e.g. sodium, potassium or calcium chloride, and the like. Examples of liquid carriers are syrup, peanut oil, olive oil, phospholipids, fatty acids, fatty acid amines, polyoxyethylene and water. Aqueous liquid formulations, in particular, may advantageously contain a non-ionic surfactant, e.g. a polysorbate [such as polysorbate 20 (e.g. Tween™ 20)] or a poloxamer [such as poloxamer 188 (e.g. Pluronic™ F68) or poloxamer 407 (e.g. Lutrol™ F127)], and a preservative, such as benzyl alcohol, phenol or a cresol (e.g. m-cresol), will often be incorporated.

It may be advantageous to provide hGH in the form of a sustained release formulation. As such, the composition may be formulated as microcapsules or microparticles containing the growth hormone encapsulated in, or dispersed in, a suitable pharmaceutically acceptable biodegradable polymer, such as polylactic acid, polyglycolic acid or a lactic acid/glycolic acid copolymer.

Recombinantly produced hGH (22 kDa hGH) has been commercially available for a number of years. Its use for therapeutic purposes is preferred, owing—among other reasons—to the fact that preparations prepared from human pituitary (from cadavers) could contain infectious agents, such as the causative agent of Creutzfeldt-Jacob's disease.

A number of recombinant hGH (22 kDa hGH) preparations are commercially available and are suitable for therapeutic use in the context of the present invention. Suitable liquid formulations of hGH (suitable, e.g., for administration by injection) include, for example, Norditropin™ SimpleXx™ (Novo Nordisk A/S), whilst suitable freeze-dried hGH formulations (for reconstitution in an appropriate liquid medium) include Norditropin™ (Novo Nordisk A/S).

When administering hGH per se in accordance with the invention, a suitable dose will frequently be in the range 50-75 μg hGH/kg body weight/day, such as in a range of 50-70 or 55-65 μg hGH/kg body weight/day [e.g. in the form of a single daily dose containing 50-70 (e.g. 55-65) μg hGH/kg body weight]. On the basis of results obtained in connection with the treatment of tibia fractures (vide infra), an optimal dose level for hGH in connection with methods according to the invention appears to a dose of about 60 μg hGH/kg body weight/day (e.g. suitably in the form of a single daily dose of about 60 μg hGH/kg body weight). These dose levels are likewise believed to be appropriate when employing, for example, Met-hGH instead of hGH per se in a method according to the invention.

It may be mentioned here that the amount of hGH polypeptide present in a dosage unit is sometimes specified in the form of “International Units” (IU). In the context of the present invention, a quantity of 1 IU of hGH in contemporary preparations of hGH may normally be taken to be equivalent to 350 μg hGH (i.e. such that 100 μg hGH corresponds to 0.286 IU hGH).

In relation, in particular, to the possibility of employing a formulation of hGH, or of an analogue or derivative thereof, possessing sustained-release properties in treatment of long-bone fractures, the feasibility of employing local application of such a formulation (i.e. application to, or in the immediate vicinity of, the fracture), instead of subcutaneous administration in the manner presently discussed and claimed herein, should be taken into consideration [see, e.g., Andreassen et al., Calcified Tissue International, 73, 258-264 (2003)]. Dosage levels appropriate for such a treatment are presently difficult to estimate, but a dosage level for such a formulation equivalent to 0.25-3 mg hGH per day is believed to be realistic.

The present invention encompasses the possibility of administering hGH, or an analogue or derivative thereof, in combination with one or more additional therapeutically active agents or substances that may be able to provide some additional therapeutic benefit to the treated human subject in the context of the present invention, e.g. with respect to promoting healing of a long-bone fracture in the manner of the invention. However, important embodiments of the methodology of the invention employ hGH, or an analogue or derivative thereof, as the sole therapeutically active agent. By way of example, a substance such as a growth hormone secretagogue (GHS), or another growth-promoting agent or growth factor (i.e. other than hGH per se, or analogues or derivatives of hGH), will not normally be administered to the human subject in conjunction with administration of hGH, or an analogue or derivative thereof, in the manner of the invention. Thus, for example, in numerous embodiments, agents such as vascular endothelial growth factor (VEGF), insulin-like growth factor I (IGF-1), growth hormone releasing hormone (GHRH), thrombopoietin (TPO) or erythropoietin (EPO), or other growth-related factors [such as leukaemia inhibitory factor (LIF) or ciliary neurotropic factor (CNTF)], will not normally be administered together with hGH or an hGH analogue or hGH derivative in the context of the present invention.

In certain embodiments of the methodology of the invention, hGH or an hGH analogue may be delivered to the subject to be treated via expression of an exogenous nucleic acid sequence coding for the hGH or hGH analogue (e.g. comprised in a plasmid or viral vector), which is administered to the subject under conditions suitable for expression of the hGH or hGH analogue at dosages sufficient to promote healing of the long bone fracture.

Administration of hGH, or of an analogue or derivative thereof, in accordance with the invention normally takes place until clinical healing (as assessed by the investigator in question) of the fracture has occurred. Different investigators will often have rather different criteria for assessing clinical healing of a fracture (including, e.g., the patients ability to bear weight on the fracture, extent of pain, general appearance of the fracture in X-ray photographs).The extent of healing of a bone fracture is, however, more reliably evaluated radiographically, and an appropriate radiographic criterion for fracture healing in the present context (referred to hereafter as the central radiographic criterion) is the presence—determined radiographically—of cortical bridging in 3 out of the 4 bone cortices and/or complete disappearance of the fracture lines. Fracture healing assessed on a clinical basis will often have occurred earlier (e.g. 1-2 months earlier) than fracture healing as assessed on the basis of the central radiographic criterion. In the study described herein (vide infra), clinical healing of long-bone fractures in connection with treatment thereof according to the invention was frequently achieved within a period of 16 weeks or less.

As already indicated to some extent above, treatment of long-bone fractures in accordance with the invention is normally carried out in conjunction with preceding primary fixation of the fractured long bone using one or more of a variety of techniques which will be well known to medical personnel of ordinary skill in the field of bone fracture treatment. These fixation techniques may be divided into non-surgical and surgical techniques. Non-surgical fixation techniques include so-called closed reduction, plaster casting, traction techniques and the like. Surgical fixation techniques include internal and external techniques. Internal fixation techniques are techniques which achieve fixation by the use of fixation means such as wires, screws, plates, nails, pins and the like applied on or within the bone itself, or by the use of a combination of two or more such fixation means. Such internal fixation means span the fracture site and are in close contact therewith. An example of a well known internal fixation technique of rather general applicability in this connection is so-called intramedullary nailing or pinning, in which a stiffening means such as a rod, nail or pin is inserted within the bone itself, i.e. in the intramedullary canal, so as to span the fracture site. External fixation techniques are techniques in which the fracture is stabilised by percutaneous insertion of pins or screws in the bone on both sides of the fracture site, after which a stiffening bar, rod or the like is attached externally (i.e. outside the skin) thereto so as to join the inserted pins or screws. Such external fixation techniques are well known to medical personnel of ordinary skill in the field of bone fracture treatment, and examples thereof include fixation in accordance with the Hoffmann, Ilizarow or Ortofix methods.

A further aspect of the present invention relates to a method for promoting the sale or use of hGH, or of an hGH analogue or hGH derivative, or any combination thereof, comprising distributing information (whether by print, radio, television, e-mail, internet advertising, promoted lectures with key opinion leaders, in-person meetings with scientific/medical liaisons, mass mailings, etc.) promoting the use of hGH, or of an hGH analogue or hGH derivative, or any combination thereof, in the treatment of long-bone fracture in a subject. The information promoting the sale or use of hGH, or of an hGH analogue or hGH derivative, may describe or specify any of the aspects or embodiments of the invention as set forth above.

### BRIEF DESCRIPTION OF THE DRAWINGS

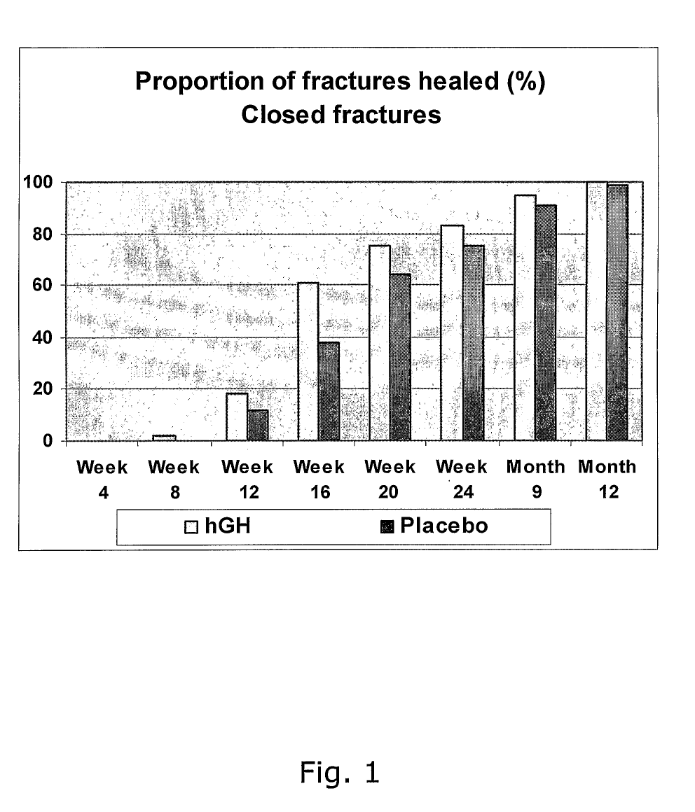

FIG. 1 shows data for healing of closed fractures during 12 months.
hGH dose 60 μg/kg body weight;
all fractures: n.s.;
closed fractures: p=0.026;
median healing time in closed fractures: hGH 95 days; placebo 126 days.

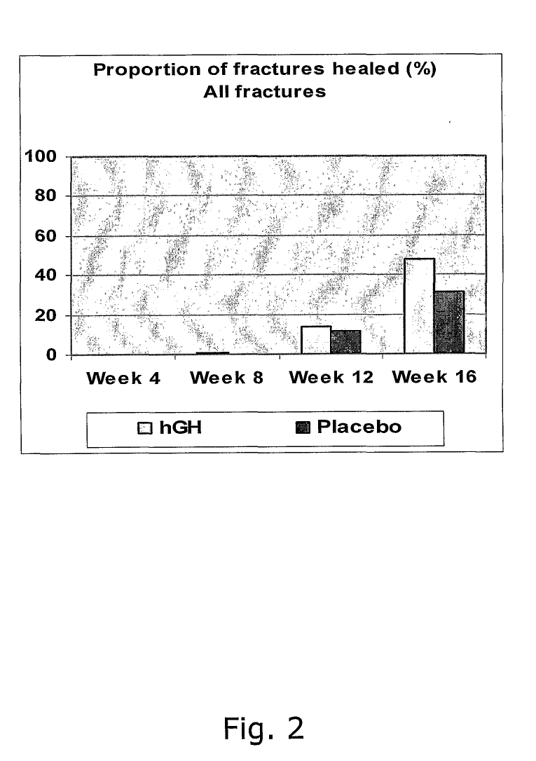

FIG. 2 shows data for fracture healing following max. 16 weeks treatment (all fractures).
hGH dose 60 μg/kg body weight;
all fractures: p=0.020;
closed fractures: p=0.042;
no safety concerns.

### EXPERIMENTAL SECTION

### Study:

A randomised, multi-centre, double-blind, placebo-controlled, parallel group trial investigating the efficacy and safety of three dosage levels of human growth hormone (recombinant 22 kDa hGH) in treatment of tibia fractures was performed. A total of approx. 400 patients having closed or open tibia fractures participated in the study, and were divided (randomized) into three treatment groups and one placebo group, each consisting of approx. 100 patients (vide infra). The hGH [in the form of Norditropin™ SimpleXx™ (Novo Nordisk A/S) cartridges containing 10 mg hGH pr. 1.5 ml] was administered as a subcutaneous injection once daily to patients in the three treatment groups at a dose of 15 μg hGH/kg body weight (Group 1), 30 μg hGH/kg body weight (Group 2) and 60 μg hGH/kg body weight (Group 3), respectively. Patients in the placebo group (Group 4) received a solution having the same composition as Norditropin™ SimpleXx™ but lacking hGH. In this study, treatment was given for a maximum of 16 weeks [or until clinical fracture healing (vide infra), if such healing had occurred before 16 weeks].

Treatment with Norditropin™ SimpleXx™/Placebo was initiated with a low dose, increasing gradually in order to minimise the occurrence of adverse events such as water retention, until the appropriate dose for the particular patient was reached, as follows:

Daily dose [μg hGH/kg body weight (bw)] of
Norditropin ™ SimpleXx ™

| | | | Week 3 |
|---|---|---|---|
| Treatment | | | (final |
| Group | Week 1 | Week 2 | dose) |
| Group 1 | 5 | 10 | 15 |
| (15 μg/kg | | | |
| bw) | | | |
| Group 2 | 10 | 20 | 30 |
| (30 μg/kg | | | |
| bw) | | | |
| Group 3 | 20 | 40 | 60 |
| (60 μg/kg | | | |
| bw) | | | |

The maximum daily dose of hGH administered to any particular patient did not exceed 5 mg.

### Patient Inclusion/Exclusion Criteria

Key inclusion criteria were as follows:
Age≧18 years and <65 years
Primary surgical treatment of tibia fracture using intramedullary nailing
Closed fractures: Tscherne Type C1, C2 and C3
Open fractures: Gustilo Grade I, II and IIIa.

Key exclusion criteria were:
Pre-existing bone and/or soft tissue infection
Severe head injury (as defined by patients being stuporous or comatose with papillary enlargement or asymmetry).

### Assessment:

### Primary Efficacy Endpoint:

Time from surgery until fracture had healed.

Fracture healing was assessed on the basis of X-ray radiographic evaluation (as outlined earlier, above).

### Secondary Efficacy Endpoints:

Investigator's assessment: fracture healed (yes/no) based on radiographic examination and physical examination
Callus index around the fracture site assessed on standard radiographs in two planes at the point in time at which the fracture has healed. Callus index is defined as the ratio of the maximum width of callus to the diameter of the original bone shaft at the same level.
Number of required secondary procedures related to the fracture (replacement of intermedullary nail, bone graft, dynamisation)
Number of fractures healed.

### Safety Endpoints:

Functional complications assessed by physical examination (fracture site pain after activity, knee pain, decreased ankle motility; long-term disability)
Incidence of fracture site infection as assessed by physical and laboratory examination
Adverse events.

### Laboratory Assessments:

### Safety Assessments:

Random blood glucose measurements
Routine haematology (hemoglobin, white blood cell count, platelet count) and blood chemistry (creatinine, sodium, potassium, protein total, bilirubin, alkaline phosphatases, alanine aminotransferase (SGPT), aspartate aminotransferase (SGOT)
HbA_{1c}
Urinalysis (protein, glucose, red and white blood cells).

### Efficacy Assessments:

Serum IGF-I, IGFBP-3
Bone markers: osteocalcin, bone-specific alkaline phosphatase, C-terminal telopeptides of type I collagen (CTX).

### Other Assessments:

Quality-of-Life assessments performed using the following validated questionnaires:
   SF-36 Health Survey
   EQ-5D Health Questionnaire
Number of days at/off work
Healing of the ipsi-lateral fibula
Diary: the following were recorded:
   Date
   Dose of trial drug injected
   Adverse events.

The overall design of the trial is summarized in Scheme 1, below:

Scheme 1. Trial design tibia fractures§§
Closed (70%) and open fractures (30%)

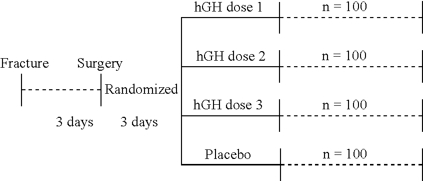

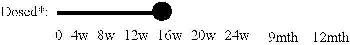

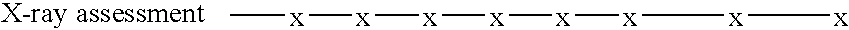

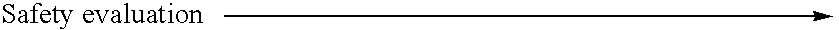

*or until clinical healing if before 16w
§§Actual numbers of patients (n) in each group were as follows: Group 1 (dose 1): 99; Group 2 (dose 2): 99; Group 3 (dose 3): 108; Group 4 (placebo): 100

### Results:

The results of the study demonstrate that treatment of acute long-bone (tibia) fractures (i.e. fractures for which a maximum of 6 days has elapsed after fracture and a maximum of 3 days has elapsed after surgery) by once-daily subcutaneous (s.c.) administration of 60 μg hGH/kg body weight for a duration of up to 16 weeks accelerates healing significantly in a period of up to 12 months post-surgery compared to standard-of-care (SOC) treatment. A dose of 60 μg hGH/kg/day resulted in marked acceleration of fracture healing compared to placebo treatment, whereas treatment with the lower doses of 15 and 30 μg hGH/kg/day resulted in no significant improvement relative to placebo. This result is surprising, since the levels of endogenous markers of hGH activity (IGF-I, IGFBP-3, Osteocalcin (bone)) in all three hGH-dosed groups were significantly increased compared to placebo. This may possibly indicate that a minimum dose level in the vicinity of 60 μg hGH/kg body weight/day is necessary in order to obtain sufficient stimulation of the autocrine and/or paracrine release of growth factors in bone (IGF-I etc.) and in other fracture-related tissues that can induce acceleration of fracture healing. In addition, the study also revealed that treatment of long-bone (tibia) fractures with 60 μg hGH/kg body weight/day administered s.c. significantly reduced the number of days off-work (sick leave days) experienced by the patients, indicating that treatment in the manner of the invention can result in an earlier return to normal daily work/activities compared to SOC treatment.

The major results of the study are summarized in FIGS. 1 and 2 and Schemes 2-4, below. The fracture healing results summarized in FIGS. 1 and 2 and Scheme 2 are based on application of the radiographic criteria for fracture healing which are discussed earlier, above, rather than clinical criteria.

Scheme 2. Healing time in open fractures
compared to closed fractures^{##}

| Proportion of fractures | | |
|---|---|---|
| healed at 16 weeks | Placebo | hGH |
| Closed | 38% | 61% |
| Open | 12% | 26% |

^{##}hGH dose 60 μg/kg body weight

Scheme 3. Number of days off work (open fractures)
Open fractures, Days off work, p = 0.038

| Group | Placebo | | 15 μg/kg | 30 μg/kg | 60 μg/kg |
|---|---|---|---|---|---|
| ITT analysis set | N | 23 | 23 | 25 | 33 |
| Completers | N | 19 | 16 | 17 | 30 |
| Mean (days) | | 189 | 181 | 169 | 130 |
| Median (days) | | 172 | 144 | 166 | 112 |
| More than 180 days (%) | | 47 | 31 | 47 | 20 |

Scheme 4. Number of days off work (closed fractures)
Closed fractures~More than one fracture line
Days off work, n.s.

| Group | Placebo | | 15 μg/kg | 30 μg/kg | 60 μg/kg |
|---|---|---|---|---|---|
| ITT analysis set | N | 37 | 49 | 41 | 39 |
| Completers | N | 35 | 47 | 32 | 33 |
| Mean (days) | | 152 | 151 | 127 | 133 |
| Median (days) | | 112 | 140 | 112 | 112 |
| More than 180 days (%) | | 23 | 23 | 16 | 12 |

It is apparent from the above results and discussion that the methodology of the present invention makes an important contribution to meeting a long-felt need for improving the healing prognosis and healing outcome of long-bone fractures in adult human patients. In addition, the methodology is able to achieve a significant economic contribution in reducing the number of off-work days (sick days) experienced by patients.

All references, including publications, patent applications, and patents, cited herein are hereby incorporated by reference to the same extent as if each reference were individually and specifically indicated to be incorporated by reference and were set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The use of the terms “a” and “an” and “the” and similar referents in the context of describing the invention (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element to be essential to the practice of the invention.

The citation and incorporation of patent documents herein is done for convenience only and does not reflect any view of the validity, patentability, and/or enforceability of such patent documents.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a method described herein as comprising a particular step should be understood as also describing a method consisting of that step, unless otherwise stated or clearly contradicted by context).

This invention includes all modifications and equivalents of the subject matter recited in the aspects presented herein to the maximum extent permitted by applicable law.

1. A method of promoting healing of a long-bone fracture in a human subject in need of such treatment, comprising administering human growth hormone (hGH), an analogue thereof, or a derivative of either thereof, subcutaneously to said subject in a dose equivalent to 45-10 μg hGH/kg body weight/day.

2. The method according to claim 1, wherein said administration of hGH, analogue, or derivative thereof, takes place once daily.

3. The method according to claim 1, wherein said subject is an adult human.

4. The method according to claim 3, wherein said long-bone fracture is an acute or sub-acute fracture.

5. The method according to claim 3, wherein human growth hormone (hGH) is administered to said subject.

6. The method according to claim 5, wherein hGH is administered in a dose of 50-70 μg/kg body weight/day.

7. The method according to claim 6, wherein hGH is administered in a dose of about 60 μg/kg body weight/day.

8. The method according to claim 3, wherein said long-bone fracture is an open or closed fracture of the femur, the tibia, or the fibula of a leg.

9. The method according to claim 8, wherein said long-bone fracture is an open or closed tibia fracture.

10. The method according to claim 3, wherein said long-bone fracture is an open or closed fracture of the humerus, the ulna, or the radius of an arm.

11. The method according to claim 7, wherein hGH is administered as a single daily dose until clinical fracture healing is achieved.

12. The method according to claim 5, wherein hGH is administered as a single daily dose until fracture healing as assessed by the central radiographic criterion is achieved.

13. The method according to claim 1, further comprising fixation of the fractured long bone using one or more internal or external fixation techniques.

14. The method according to claim 13, wherein said internal fixation technique is intramedullary nailing.

15. The method according to claim 1, wherein hGH or an hGH analogue is delivered to said subject by expression of an exogenous nucleic acid sequence which codes for hGH or said hGH analogue, and which is administered to the subject under conditions suitable for expression of hGH or said hGH analogue at dosages sufficient to promote healing of said long-bone fracture.

16-31. (canceled)
